(19) United States
(12) Patent Application Publication
(10) Pub. No.: US 2015/0297148 A1
(43) Pub. Date: Oct. 22, 2015

(54) Magnetic Resonance Facility with a Display Apparatus

(71) Applicants: Stephan Biber, Erlangen (DE); Swen Campagna, Engelthal (DE); Stephan Nufer, Erlangen (DE); Thorsten Speckner, Erlangen (DE); Jens Thöne, Nurnberg (DE)

(72) Inventors: Stephan Biber, Erlangen (DE); Swen Campagna, Engelthal (DE); Stephan Nufer, Erlangen (DE); Thorsten Speckner, Erlangen (DE); Jens Thöne, Nurnberg (DE)

(21) Appl. No.: 14/687,301

(22) Filed: Apr. 15, 2015

(30) Foreign Application Priority Data
Apr. 16, 2014 (DE) 102014207295.6

(51) Int. Cl. A61B 5/00; A61B 5/055

(52) U.S. Cl. CPC A61B 5/7445; A61B 5/055

(57) ABSTRACT

A magnetic resonance facility has a main magnet unit with a cylindrical patient accommodation region, and an optical display apparatus with at least one display unit disposed within the patient accommodation region in a region viewable by a patient positioned therein for the magnetic resonance measurement. The display apparatus is shaped at least partially to follow the boundary of the patient accommodation region.

This application claims the benefit of DE 102014207295.6, filed on Apr. 16, 2014, which is hereby incorporated by reference in its entirety.

### FIELD

The disclosed embodiments relate to a magnetic resonance facility having a main magnet unit with a cylindrical patient accommodation region.

### BACKGROUND

Magnetic resonance imaging is well known in the medical field and is now a widely used imaging method. In order to be able to generate powerful and homogeneous magnetic fields, magnetic resonance facilities frequently have a structure, in which a cylindrical patient accommodation region is provided in a main magnet unit, into which the patient may be moved via a patient couch. Because the gradient coil arrangement and a high-frequency coil arrangement are also arranged to enclose the patient accommodation region, the patient accommodation regions, often also referred to as bores or tunnels, have a very small diameter, within which a powerful magnetic field is present, the homogeneity of which may be impaired in the imaging region.

Because magnetic resonance measurements, in other words magnetic resonance image recordings, frequently also take a long time, the restricted patient accommodation region, which may have a diameter of 60 or 70 cm for example, may be problematic. Patients frequently find the patient accommodation region restricting and therefore unpleasant, which may result in discomfort on the part of the patient and spontaneous movement/blurred magnetic resonance recordings. Magnetic resonance recordings affected by motion artifacts have to be repeated, thereby extending the overall examination time. Patients with a tendency to claustrophobia are more badly affected by the restricting nature of the patient accommodation region and they may even refuse to be examined using the magnetic resonance facility.

It has been proposed that claustrophobic patients should be given sedatives. But this proposal is not desirable, as it may affect the patient adversely, producing for example side effects, such as drowsiness and loss of motivation, and resulting in the patients not being allowed to operate machinery or drive motor vehicles.

Patients in the patient accommodation region also easily lose any sense of time due to the unpleasant situation. The patients are unable to gauge how much longer the magnetic resonance measurement still has to run, unless the person performing the examination actively informs them of the progress of the examination, for example, via an intercom system.

It is largely only possible to communicate acoustically with the patient. The use of a mirror on the patient couch has been proposed for visual communication, for functional magnetic resonance at any rate, in order to convey stimuli. The mirror reflects the image of an external display apparatus.

Until now there has been no known effective solution to the impact of the restricting nature of the patient accommodation region. Better examination results have been achieved as a result of better cooperation on the part of the patient due to prior instruction, for example, the showing of videos relating to the examination process. The restricted feeling has been reduced by distraction measures, for example music, but these measures are complex and may still be improved upon.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, the disclosed embodiments may provide a magnetic resonance facility configured such that the feeling of restriction in the patient accommodation region may be counteracted while supporting improved communication with the patient.

In one aspect, an optical display apparatus with at least one display unit is disposed within the patient accommodation region in a region that may be perceived (viewed) by a patient positioned therein for the magnetic resonance measurement. The display apparatus is shaped at least partially to follow the boundary of the patient accommodation region.

Information/images are displayed within the patient accommodation region, to which end a curved display apparatus, or a curved video surface, which may be integrated in the patient accommodation region, is provided. The curved display apparatus therefore does not restrict or only very slightly restricts the patient accommodation region because of the specific shape that follows the boundary of the magnetic resonance accommodation region. Technologies may be used for producing curved display apparatuses, which may also function in powerful magnetic fields. In the event that the display apparatus affects the magnetic field itself, in a manner that is relevant to imaging, provision may be made for such to be taken into account in the design of gradient coil arrangements, high-frequency coil arrangements and shim coil arrangements, shaping the effective field in the imaging region correspondingly. Corresponding methods for determining conductor patterns and the like may be used to obtain a desired field distribution.

The at least one display unit allows the patient to be distracted so that he/she perceives the restricting nature of the patient accommodation region as less disturbing. For example, a feeling of spatial depth may be produced from the point of view of the patient via a video signal, which in turn may lead to a decreased feeling of restriction and anxiety for claustrophobic patients. The display apparatus may also be used to inform the patient and to output instructions to the patient. The magnetic resonance facility is useful in the field of functional magnetic resonance imaging as, compared with the former mirror solutions for conveying optical stimuli, the integrated display in the magnetic resonance accommodation region is much less susceptible to error and much less complex. On the one hand, the mirror and external display apparatus do not have to be adjusted. on the other hand, the external device is completely omitted from the examination space, including the cabling and other components of the external device.

In one embodiment, the display apparatus includes at least in part a number of display units arranged in the manner of a matrix, with display units of different matrix rows being arranged at an angle to one another to provide shape. The display apparatus may be made up at least partially or completely of small display units, as macroscopically done for example for monitor walls and the like. Because different rows of display units are arranged at an angle to one another, with the angle between all the matrix rows optionally being identical in the case of a cylindrical embodiment of the magnetic resonance accommodation region, the cylindrical shape of the magnetic resonance accommodation region may be recreated, with the accuracy of the recreation and therefore also the ability to save space being a function of the size of the display units in the column direction of the matrix.

Fewer or just a single display unit may be used within the display apparatus, the display(s) already being essentially matched to the shape of the boundary of the patient accommodation region and/or being able to be matched thereto, for example due to flexibility. The display apparatus may thus have at least one display unit, the shape of which is matched and/or may be matched to the boundary of the patient accommodation region. The shape-matched display unit may be implemented on a curved substrate. Circuit boards that may be produced in any shape are already known, so in such an instance the display unit may be manufactured from sufficiently small structural elements that it may also be implemented in a curved manner.

The shape-matchable display unit may be implemented on a flexible substrate. Organic light-emitting diodes (OLEDs) may be used for such an embodiment, in which the shape of the display units is therefore not fixed. The shape-matched and/or shape-matchable display unit may be a display unit based on organic light-emitting diodes having a number of organic light-emitting diodes. Such OLED display units may be useful in that the display units allow a capacity for movement of the substrate thereof, and therefore attain a certain flexibility, as proposed for example in connection with electronic paper implemented via OLEDs. The flexible substrate may be a flexible circuit board. A material that may be obtained and processed in a favorable manner may be used for the flexible substrate, such as, for example, PET.

The use of OLED technology therefore allows display units to be produced in a straightforward manner, being able to be or being already matched to the cylindrical shape of the patient accommodation region. Such OLED display units may also function in powerful magnetic fields and may be implemented so that the units are as magnetic resonance-compatible as possible.

The display apparatus may include a layer of protective material at least in the region of the outer face of the at least one display unit. Such a protective material or surface material may be robust, scratch-resistant and/or sterilizable. The protective material may also be high frequency (HF)-transparent and/or biocompatible. The face of the at least one display unit facing toward the interior of the patient accommodation region may therefore expediently be configured from a durable, scratch-resistant, biocompatible, HF-transparent material, in order to allow implementation to be technically compatible with the electromagnetic and other characteristics of the patient accommodation region. For example, with respect to magnetic resonance compatibility, technologies used in magnetic resonance PET facilities may be used, where a larger number of further electronic components, e.g., the photon detectors, are also located within the patient accommodation region.

Plexiglas, glass or a thermoplastic polycarbonate may be used as protective materials. Thermoplastic polycarbonates, e.g., LEXAN materials, may be suitable for coating or being applied in a protecting manner to display units in the magnetic resonance facility.

A high-frequency coil arrangement may be provided to enclose the patient accommodation region, e.g., following a gradient coil arrangement in an inward direction, with the display apparatus integrated therein or fastened thereto. An integrated embodiment may be provided. For example, the display apparatus may be implemented as part of a support tube of the high-frequency coil arrangement and/or may be fastened to the support tube. While the display apparatus may also be positioned on the cladding of the patient accommodation, which may reduce the space available for the patient, in one embodiment the display apparatus is configured in an integrated manner with the high-frequency coil arrangement (body coil, or BC), so that the standard internal cladding of the patient accommodation region may be fitted around the display apparatus.

The cladding may be provided in segments of the internal boundary of the patient accommodation region not provided as display regions of the display apparatus. In one embodiment, the cladding and the display apparatus or its display regions, as defined by the display units, adjoin one another in a flush manner, forming a continuous, smooth surface. The display apparatus therefore becomes a seamless part of the overall impression of the patient accommodation region, replacing the internal cladding at some points, so no additional internal space is required.

The display apparatus may be activated by a control facility. A central control facility of the magnetic resonance facility may also be used to activate the display apparatus and therefore to determine the images to be shown on the display apparatus. The data transmission and/or energy transmission to the display apparatus may occur in a wired manner. But as data transmission and/or energy transmission may be performed wirelessly within a magnetic resonance facility, such transmission may be used for the display apparatus, so no additional cabling is involved.

The control facility may be configured to show an image conveying spatial depth and/or at least one item of patient information relating to a magnetic resonance measurement to be performed and/or a visual stimulus when performing a functional magnetic resonance measurement. The display apparatus may therefore be used to show a patient images, for example, digital videos and/or photographs, which convey an impression of spatial depth and/or have a calming effect. For example, a patient may be shown calming scenes, such as an animated sky and/or a sea view, during the magnetic resonance measurement.

Patient information may also be displayed in addition to the described images. At least the remaining duration of the magnetic resonance measurement and/or the fraction of the magnetic resonance measurement already completed and/or an instruction relating to breathing to be able may be output as patient information. Superimposition of the examination duration or the progress of the examination, e.g., as a progress indicator, such as a bar chart, a progress bar or the like, constantly provides the patient with information about how long the examination still has to run, thereby giving the patient a sense of progress and restoring any lost sense of time. The display apparatus or at least one of the at least one display units may be used to output instructions to patients, e.g., breathing instructions such as “hold your breath” or “breath again”. This provides an additional possible mechanism of communicating with the patient, which may render superfluous audio communication, which is sometimes more complex, e.g., because of the noise level.

The display apparatus in the patient accommodation region is also suitable for outputting visual stimuli when performing a functional magnetic resonance (fMRI) measurement. During fMRI examinations, the additional video surface is therefore used to allow the visual stimulation of patients and to replace current mirror and projection surface structures behind the patient accommodation region.

### BRIEF DESCRIPTION OF THE DRAWINGS

FIG. 1 shows a magnetic resonance facility in accordance with one embodiment.

FIG. 2 shows a simplified view of a cross section through a patient accommodation region in accordance with one embodiment.

FIG. 3 shows an exemplary display on a display apparatus in the magnetic resonance facility in accordance with one embodiment.

### DETAILED DESCRIPTION

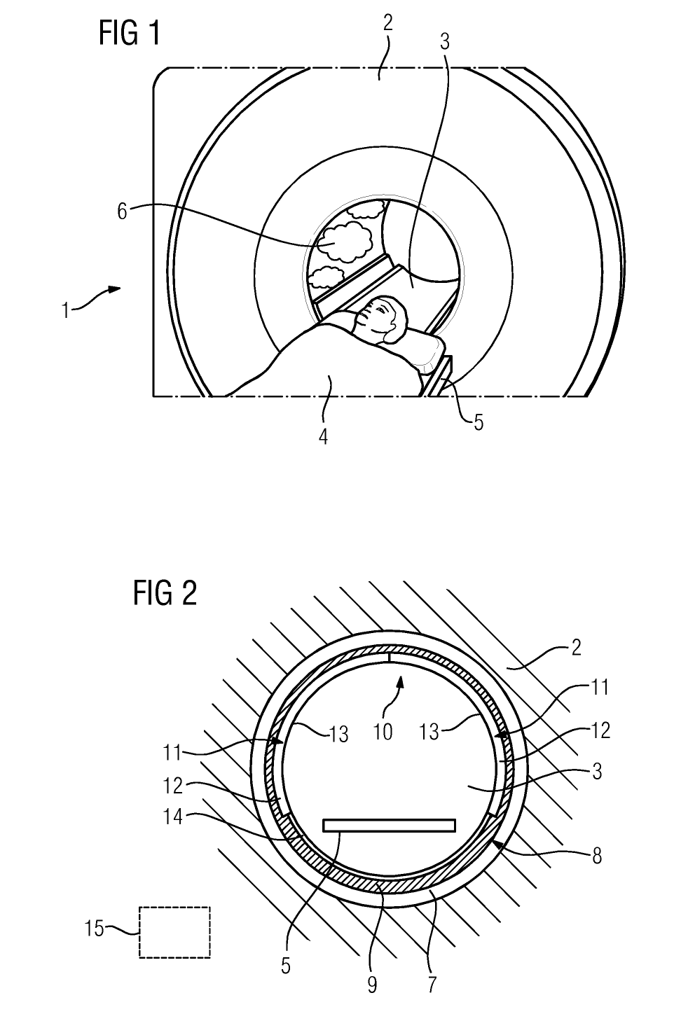

FIG. 1 shows a simplified view of an inventive magnetic resonance facility 1. The facility 1 includes a main magnet unit 2, which defines a cylindrical patient accommodation region 3, into which a patient 4 may be moved via a patient couch 5 for the magnetic resonance measurement. It may be seen that part of the internal surface of the patient accommodation region 3 is taken up by a display surface 6 of a display apparatus, on which animated clouds are shown as an image, conveying an impression of spatial depth for a patient 4 and also having a calming effect.

The display apparatus may be implemented on different small display units, which are arranged in the manner of a matrix in rows, each arranged at an angle to one another.

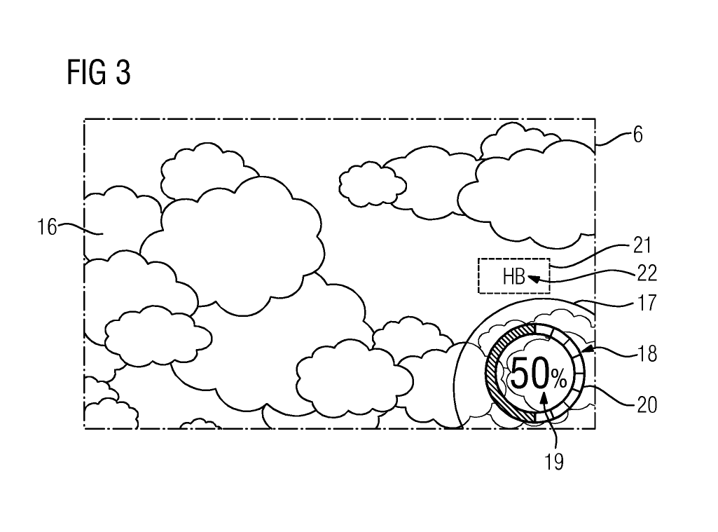

FIG. 2 shows an embodiment based on a section through the main magnet unit 2 in the region of the patient accommodation region 3. Provided to enclose the patient accommodation region are a gradient coil arrangement 7 (not shown in detail) and, following the arrangement 7 in an inward direction, a high-frequency coil arrangement 8, the support tube 9 of which is shown in detail. The display apparatus 10 in this case includes two display units 11 (shown in cross section), which are integrated in the support tube 9 or fastened in a recess and include organic light-emitting diodes (not shown in detail) disposed on a flexible substrate 12. For protection, the display units 11 are also covered with a layer 13 of protective material, which may be for example glass, Plexiglas or a thermoplastic carbonate. The substrate 12 may also be made of PET. The layer 13 is configured as scratch-resistant, biocompatible and high frequency-transparent, with the display units 11 overall also being configured to be as magnetic resonance-compatible as possible. Data transmission and energy transmission to the display units 11 of the display apparatus 10 occur wirelessly in this instance but may occur in a wired manner. The display units 11 may not cover the entire length of the patient accommodation region 3, but a number of the flexible OLED display units 11 may be provided over the length. A matrix-type arrangement of a number of display units may therefore also be provided.

The flexibility of the substrate 12 and therefore of the entire display unit 11 is such that the substrate 12 and the display unit 11 match the boundary of the magnetic resonance accommodation region 3 perfectly, with part of the cylinder of the internal surface of the patient accommodation region 3 being formed by the layer 13, which is supplemented by suitable cladding elements 14, which are shown as adjoining the display units 11 in a flush manner.

The substrates 12 may be provided pre-shaped for the boundary of the magnetic resonance accommodation region 3, this also being supported by OLED technology. The support tube 9 may not be changed, and the display apparatus 10 may be fastened on the support tube 9 or on fully circumferential cladding 14 of the patient accommodation region 3.

The operation of the display apparatus 10 is controlled by a central control facility 15 of the magnetic resonance facility 1, which therefore allows the desired information or images to be displayed. FIG. 3 shows an example of a display on the display surface 6 of the display apparatus 10 during a standard magnetic resonance measurement. The background is formed by the image 16, which in the present instance shows an animated sky. Provided in the bottom right corner of the display surface 6 is a region 17 for patient information 18 relating to a current magnetic resonance measurement. In the present instance, the display surface 6 shows a percentage indicating how much of the examination has already been completed, both in numbers 19 and via a progress indicator 20. Other useful information may also be displayed, for example, the time, the remaining duration of the magnetic resonance measurement, the name of the patient and the like.

A further region 21, which is not always used, is reserved for breathing-related instructions 22, e.g., telling the patient to hold his/her breath.

If a functional magnetic resonance measurement (fMRI) is to be performed, the display surface 6 may also display visual stimuli for the patient 4.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present invention has been described above by reference to various embodiments, it should be understood that many changes and modifications may be made to the described embodiments. It is therefore intended that the foregoing description be regarded as illustrative rather than limiting, and that it be understood that all equivalents and/or combinations of embodiments are intended to be included in this description.

1. A magnetic resonance facility comprising:
a main magnet unit comprising a cylindrical patient accommodation region;
an optical display apparatus comprising at least one display unit, the optical display apparatus being disposed within the patient accommodation region in a region viewable by a patient positioned in the patient accommodation region for a magnetic resonance measurement, the optical display apparatus being shaped at least partially to follow a boundary of the patient accommodation region.

2. The magnetic resonance facility of claim 1, wherein the optical display apparatus comprises a plurality of display units arranged in a matrix, in which display units of the plurality of display units in different matrix rows are arranged at an angle to one another to provide shape.

3. The magnetic resonance facility of claim 1, wherein the at least one display unit has a shape matched, matchable, or both matched and matchable to the boundary of the patient accommodation region.

4. The magnetic resonance facility of claim 3, wherein the at least one display unit is implemented on a curved substrate, on a flexible substrate, or on a curved and flexible substrate.

5. The magnetic resonance facility of claim 3, wherein the at least one display unit is comprises a number of organic light-emitting diodes.

6. The magnetic resonance facility of claim 1, wherein the optical display apparatus comprises a layer of protective material at least in a region of an outer face of the at least one display unit.

7. The magnetic resonance facility of claim 6, wherein the protective material is high frequency transparent, biocompatible, or both frequency transparent and biocompatible.

8. The magnetic resonance facility of claim 6, wherein the protective material is Plexiglas or glass or a thermoplastic polycarbonate.

9. The magnetic resonance facility of claim 1, wherein the patient accommodation region is configured to enclose a high-frequency coil arrangement, in which the display apparatus is integrated or to which the display apparatus is fastened.

10. The magnetic resonance facility of claim 9, wherein the display apparatus is implemented as part of a support tube of the high-frequency coil arrangement, is fastened to the support tube, or is both implemented as part of the support tube and is fastened to the support tube.

11. The magnetic resonance facility of claim 1, further comprising cladding in segments of an internal boundary of the patient accommodation region not provided as display regions of the optical display apparatus.

12. The magnetic resonance facility of claim 1, wherein the optical display apparatus is activatable by a control facility of the magnetic resonance facility.

13. The magnetic resonance facility of claim 12, wherein data transmission, energy transmission, or both data transmission and energy transmission to the display apparatus are implemented wirelessly.

14. The magnetic resonance facility of claim 12, wherein the control facility is configured to show an image conveying spatial depth, at least one item of patient information relating to a magnetic resonance measurement to be performed, a visual stimulus when performing a functional magnetic resonance measurement, or various combinations thereof.

15. The magnetic resonance facility of claim 14, wherein at least a remaining duration of the magnetic resonance measurement, a fraction of the magnetic resonance measurement already completed, an instruction relating to breathing, or various combinations thereof, are outputtable as patient information.

16. The magnetic resonance facility of claim 15, wherein the remaining duration, the fraction, or both the remaining duration and the fraction are showable as a progress indicator.

17. The magnetic resonance facility of claim 2, wherein the optical display apparatus has a shape matched, matchable, or both matched and matchable to the boundary of the patient accommodation region.

18. The magnetic resonance facility of claim 1, wherein the optical display apparatus is configured to display an image conveying spatial depth, at least one item of patient information relating to a magnetic resonance measurement to be performed, a visual stimulus when performing a functional magnetic resonance measurement, or various combinations thereof.
